(19) United States
(12) Patent Application Publication
(10) Pub. No.: US 2017/0035377 A1
(43) Pub. Date: Feb. 9, 2017

(54) NON-MAGNETIC MOBILE C-ARM FLUOROSCOPY DEVICE

(71) Applicant: John K. Grady, Harvard, MA (US)

(72) Inventor: John K. Grady, Harvard, MA (US)

(21) Appl. No.: 15/296,418

(22) Filed: Oct. 18, 2016

Related U.S. Application Data
(63) Continuation-in-part of application No. 14/259,750, filed on Apr. 23, 2014.
(60) Provisional application No. 61/927,667, filed on Jan. 15, 2014.

(51) Int. Cl. A61B 6/00; G01R 33/48; G01T 1/16; A61B 5/055

(52) U.S. Cl. CPC A61B 6/4417; A61B 6/4441; A61B 6/487; A61B 6/4405; A61B 5/055; G01R 33/4812; G01T 1/1606

(57) ABSTRACT

Applicant has disclosed an x-ray fluoroscopy machine made substantially entirely of parts which cannot be magnetized. By making the machine out of parts which cannot be magnetized, the x-ray machine can be placed in the same room as an MM machine without the fluoroscope affecting the magnetic field of the MRI machine. Both machines therefore are capable of operating (e.g., imaging) simultaneously in the same examination room. In the preferred embodiment, Applicant has disclosed making a mobile C-arm device, disclosed in U.S. Pat. No. 7,300,205, substantially out of materials which cannot be magnetized.

### RELATED APPLICATION

This application is a continuation-in-part of U.S. Nonprovisional patent application, Ser. No. 14/259,750, filed Apr. 23, 2014, which claimed priority to U.S. Provisional Patent Application, Ser. No. 61/927,667, filed Jan. 15, 2014. Applicant claims priority to those two prior applications. Applicant also incorporates by reference those prior applications in their entirety.

### FIELD OF INVENTION

This invention relates to x-ray machines. More specifically, the preferred embodiment relates to apparatus for positioning x-ray fluoroscopy units.

### BACKGROUND OF THE INVENTION

Fluoroscopy is a process for obtaining continuous, real-time images of an interior area of a patient via the application and detection of penetrating x-rays. Put simply, x-rays are transmitted through the patient and converted into visible spectrum light by some sort of conversion mechanism (e.g., x-ray-to-light conversion screen and/or x-ray image intensifier). Subsequently, the visible light is captured by a video camera system (or similar device) and displayed on a monitor for use by a medical professional. More recently, a solid-state pixelized flat panel is used for this purpose. Typically, this is done to examine some sort of ongoing biological process in the human body, e.g., the functioning of the lower digestive tract or heart.

Currently, most fluoroscopy is done using x-ray image intensifiers. These are large, vacuum tube devices (i.e., akin to a CRT or conventional television) that typically receive the x-rays in an input end, convert the x-rays to light and then electron beams, guide, accelerate, and amplify the electron beams via internal electrodes, and convert the electron beams to a minified visible image at the device's output end. An example of an x-ray image intensifier is shown in U.S. Pat. No. 5,773,923 to Tamagawa (see FIGS. 1 and 2 and accompanying description).

In designing x-ray support apparatuses, the x-ray device should ideally be positionable for use anywhere around the periphery of a patient in three dimensions (i.e., the X-, Y-, Z-planes). More specifically, it is typically desirable to utilize spherical angulation, where x-rays can be directed from any loci on an imaginary sphere centered on the patient to an isocenter of the x-ray device. (The isocenter is the point of intersection of an axis defined by the x-ray source and receptor and the axis of angulation, i.e., the axis of device rotation.) Other factors to take into account include: maintaining the x-ray beam normal to the x-ray receptor; the size of the examination room, and the room's ability to accommodate large devices; unrestricted access to the patient, especially around the head area; minimizing control complexity and/or the need for computer image correction or manipulation; and, as always, cost.

Most current x-ray device support apparatuses utilize either a parallelogram-shaped construction or a combination of C-, U-, and/or L-shaped arms for x-ray device positioning and (ideally) spherical angulation. Because parallelogram-based devices are so bulky, various C-arm based devices have been developed over the years.

C-arm fluoroscopy units have been in general use since the 1960's. Such units are used wherever a fluoroscopy image is desired, but outside the normal x-ray room. These C-arm fluoroscopy units are commonly used in surgery, for placing pacemakers, searching for foreign objects, or assisting various pain relief or orthopedic procedures. These units do this by visualizing the procedure or catheter manipulation on television x-ray screens.

There have been numerous variations in the design and construction of C-arm based x-ray gantries, but two main divisions are apparent: types where the horizontal C-arm axle comes at the patient from the left side, and types where the C-arm axle comes over the patient's head.

U.S. Pat. No. 7,300,205 to Grady (“Grady '205”) discloses a mobile C-arm support device which operates differently than the prior C-arm types identified above. That enables Grady's device to overcome angular difficulties present in prior C-arm devices.

Grady '205 discloses a portable x-ray device of the type having an x-ray source, an image receptor, and a C-arm support apparatus, wherein a C-arm slides in a stationary arc in a single X-Y plane through a C-sleeve mounted on a portable base, the improvement comprising: a rotatable tilt bearing means for pivoting the arc, and C-sleeve, in a Z-plane around a pivot axis; and a sliding pivot point means for repositioning the tilt bearing means, and pivot axis, in the X-Y plane along an arcuate path from approximately a horizontal position, when the x-ray beam is vertical, to a position approximately 60 degrees below horizontal in a vertical plane.

The design of the mobile C-arm in Grady '205 also allows for downsizing. That enables Grady's mobile C-arm to be used in small spaces, like doctors' offices with 9 foot ceilings. Grady's mobile C-arm, since smaller than prior C-arm devices, also enables a technician's unrestricted access to the patient in such small places, especially around the head area.

In recent years C-arm devices have been increasingly used in “minimally invasive” surgery to visualize the tools, actions, and results from such minimally invasive interventions, which include catheter based tools or catheter based surgical operations.

At the same time, projection x-rays cannot visualize small soft tissue objects very well, so some procedures are best done with Magnetic Resonance Imaging (“MRI”) machines which can give exquisite detail of brain tumors or lesions, or structural parts of the heart and soft organs. These items have little inherent x-ray contrast, and do not appear on projection x-rays.

The problem then remains of how to place a catheter (usable with a mobile C-arm x-ray machine) also in combination with an MRI machine, as real-time fluoroscopy is still needed to facilitate hand-to-eye coordination of catheter movement within the body, for instance, inside a blood vessel.

Modern mobile fluoroscopes have many metallic or ferromagnetic parts. Such parts would be drawn toward an MRI machine, which is exceptionally powerful, if a mobile fluoroscope was too close to the MRI machine. This leads to moving the patient (or machine) away, into a separate room, to use a mobile fluoroscope.

There is also the possibility that the magnetic field of the MRI machine will impact the x-ray system, requiring measures to protect against that problem. Such measures might include orientation of subsystems so as to be least impacted by the MRI field, or shielding sensitive parts with Mu-metal or suitably disposed ferromagnetic shielding pieces.

Mu-metal is a nickel-iron alloy, composed of approximately 77% nickel, 16% iron, 5% copper and 2% chromium or molybdenum. Mu-metal is notable for its high magnetic permeability. The high permeability makes Mu-metal useful for shielding against static or low-frequency magnetic fields.

It would be beneficial to have a mobile C-arm that would allow placing Mill catheters, or otherwise using a non-magnetic transfer table or trolley (which cannot be magnetized), ideally on a floor or ceiling track, between two indexed or measured locations so that surgical navigation data or calibration in 3D space being maintained in both imaging modalities, and in fact shared, to accomplish the medical goals.

This ancillary linear transport system or non-magnetic stretcher is obvious, and in general exists now. However, existing C-arm units cannot be brought close to the MRI machine as they would be drawn toward it immediately. They have many ferrous parts; they are simply accepted at the present time as not being useable at all with MRI, nor was that ever a design goal before this patent application.

It is therefore a primary object of the present invention to design a mobile C-arm fluoroscopy machine of minimal parts that can be magnetized, so the C-arm can be used in proximity to an MRI machine.

It is a more specific purpose to invoke use of innovative mechanical construction and materials which cannot be magnetized to allow use of a conceptually new mobile C-arm fluoroscopy machine close to an Mill machine with both the C-arm and MRI machines being capable of imaging simultaneously.

### SUMMARY OF INVENTION

Applicant has disclosed a mobile C-arm x-ray fluoroscopy machine having materials, which cannot be magnetized, to allow fluoroscopy to take place in close proximity to an MRI machine, such as in the same room. Using materials, which cannot be magnetized, to construct the x-ray machine avoids later interaction with the Mill field. Both machines are capable of imaging simultaneously in the same examination room.

### BRIEF DESCRIPTION OF THE DRAWINGS

The above objects and other advantages of the invention will become more readily apparent upon reading the following description and drawings from the U.S. Pat. No. 7,300,205, in which:

FIG. 1 is a side view of an “Angio Capable Portable X-Ray Fluoroscopy Unit with Sliding C-Arm and Variable Pivot Point,” constructed in accordance with the present invention;

FIG. 2 is another side plan view of the portable C-arm device with the slider pivot axis raised to 0 degrees (horizontal) from its FIG. 1 orientation and the C slid or translated to a vertical position;

FIG. 3 is a front plan view of the portable C-arm device at a cranio-caudal orientation;

FIG. 4 is a perspective view of the portable C-arm device in its FIG. 3 orientation;

FIG. 5 is a perspective view of the portable C-arm x-ray device with the slider pivot axis set at 0 degrees (horizontal) and the housing having been rotated, along that axis, from a vertical position (shown in phantom) to horizontal;

FIG. 6 is a perspective view of the portable C-arm x-ray device with the slider pivot axis set at 60 degrees below horizontal and the housing having been rotated from a cranio-caudal orientation (shown in phantom) at 45 degrees to 0 degrees;

FIG. 7 is a perspective view of a portable base, with a side cover removed (as in FIGS. 1 and 2), showing portions of a rotatable tilt bearing means and a sliding pivot point means inside the base; and

FIG. 8 shows the portable C-arm x-ray device in the same examination room as an MRI machine.

### BRIEF DESCRIPTION OF THE PREFERRED EMBODIMENTS

Applicant hereby incorporates by reference the following patents in their entirety: U.S. Pat. No. 7,300,205 to Grady (“Grady '205”), and U.S. Pat. No. 6,789,941 to Grady (“Grady '941”).

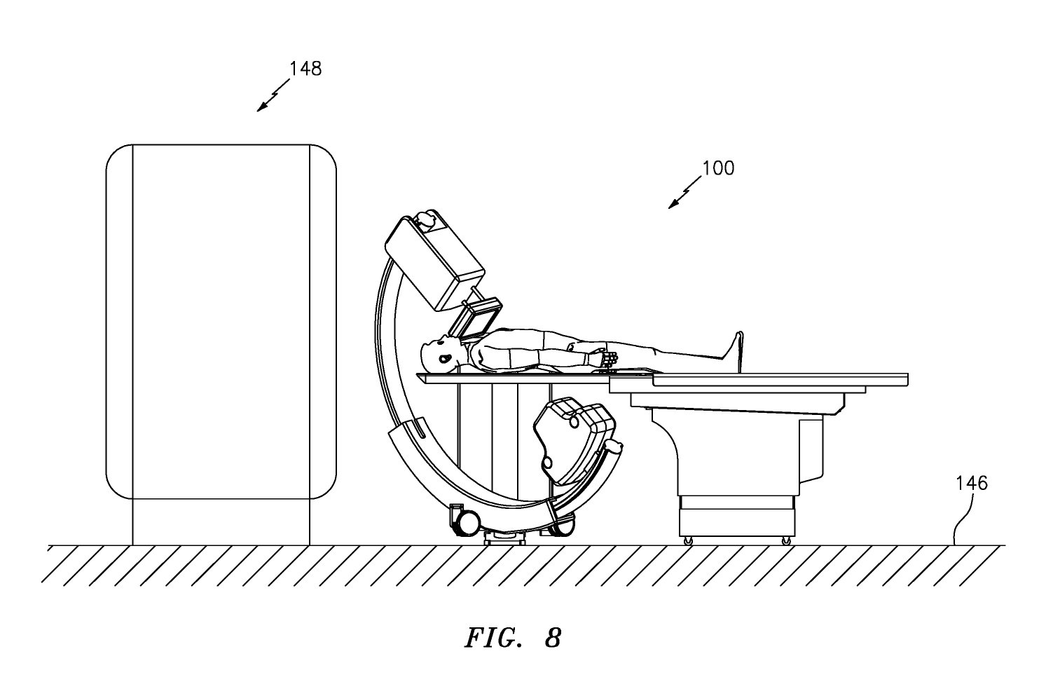

The drawings of Grady '205 show a sample mobile C-arm, which externally would not look different, except perhaps for the materials used, from the preferred embodiment of this present invention. Applicant's invention is to build a mobile C-arm x-ray fluoroscopy machine (e.g., the machine disclosed in Grady '205) substantially entirely out of materials which cannot be magnetized. That results in a new capability—interoperability of the mobile C-arm with an MRI machine in proximity to it (e.g., in the same room—see FIG. 8) without any shielding partition. Both the C-Arm fluoroscopy machine and the MRI machine are capable of operating (e.g., imaging) simultaneously, without interfering with each other, due to the C-arm machine being constructed substantially entirely out of materials which cannot be magnetized.

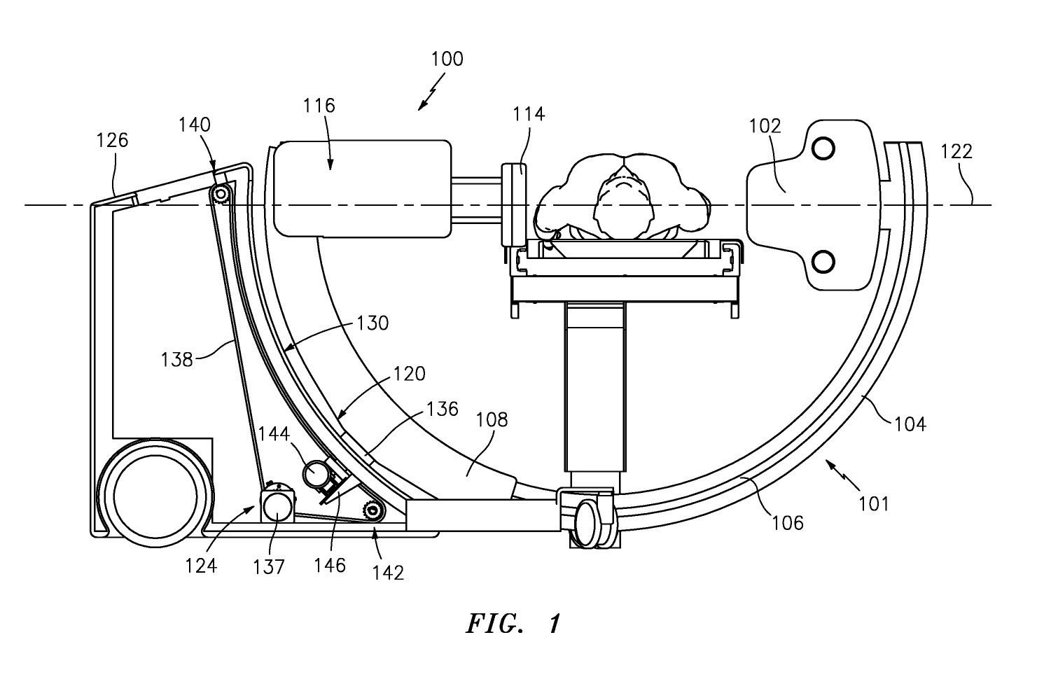

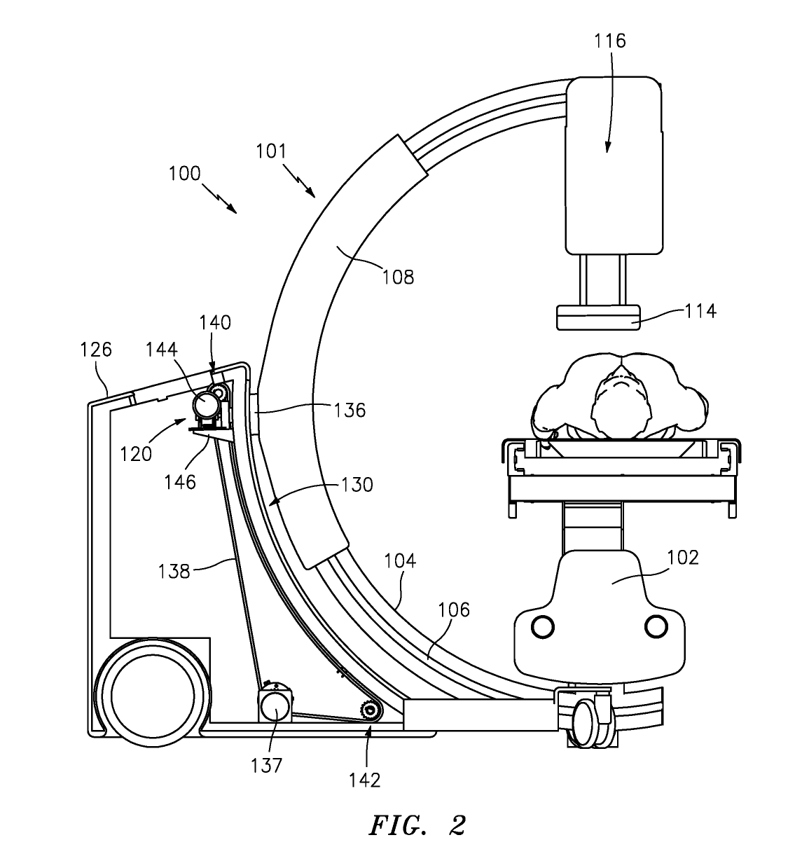

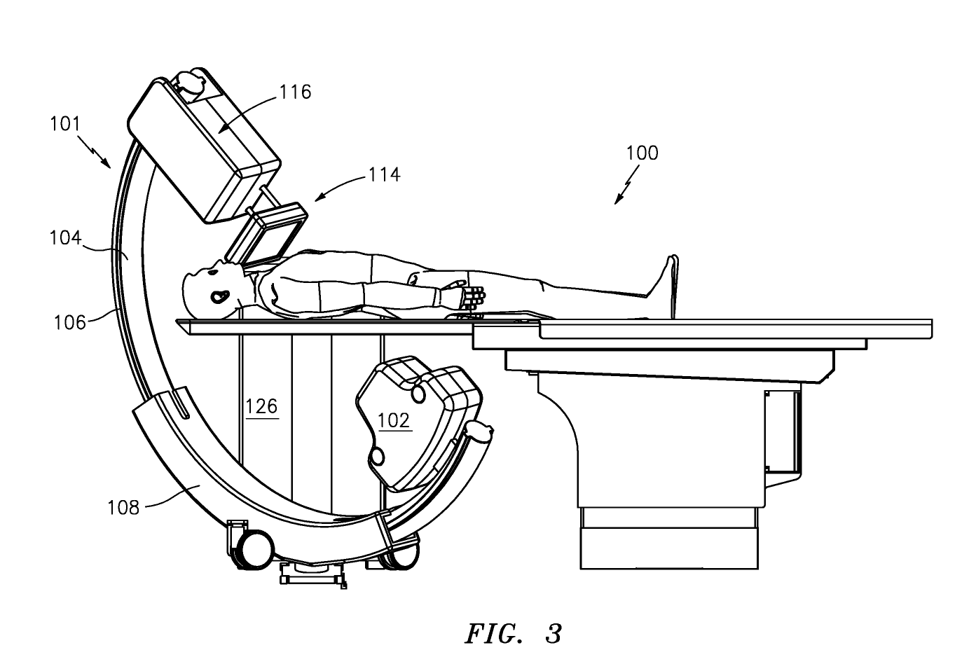

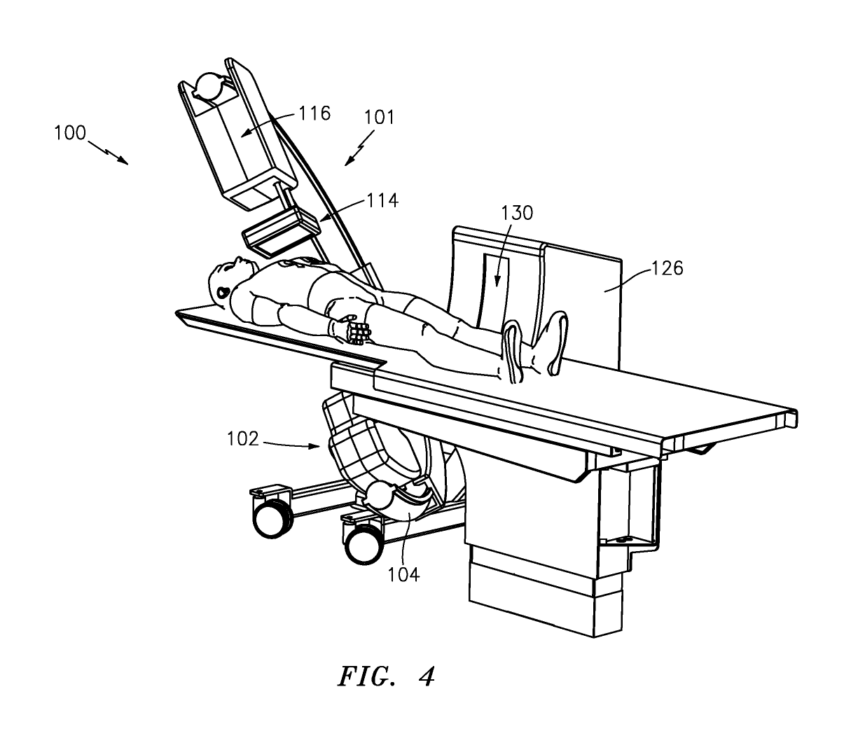

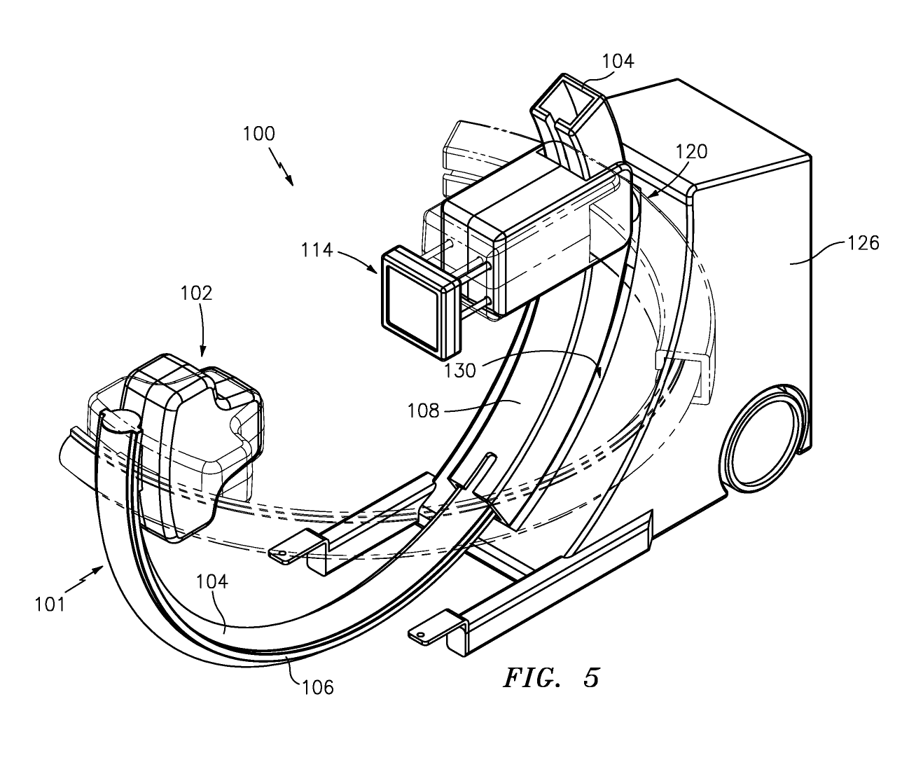

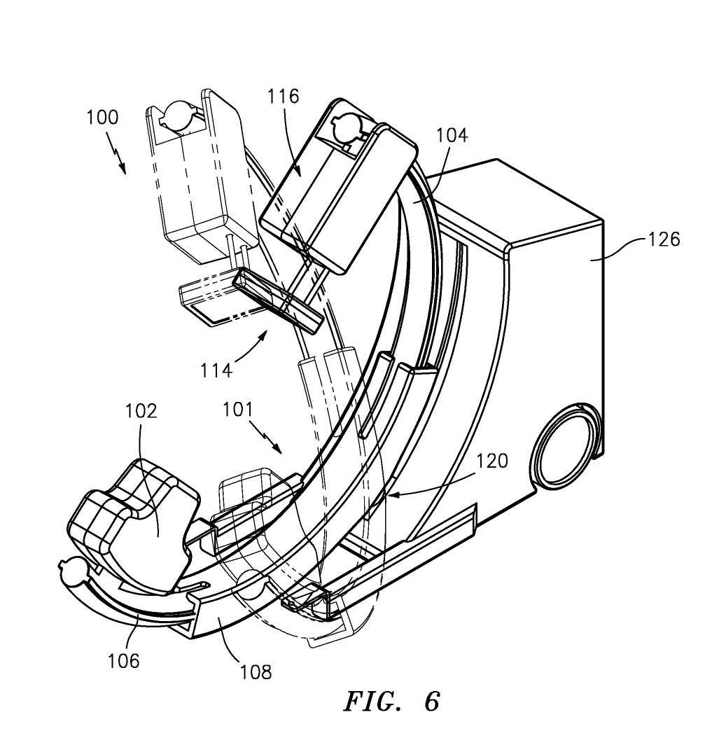

Referring to FIGS. 1-7, Applicant has disclosed a preferred embodiment of an “Angio Capable Portable X-Ray Fluoroscopy Unit with Sliding C-Arm and Variable Pivot Point” 100. The unit includes a C-arm support apparatus 101 comprising a large radius C-arm 104 having slide travel rails (e.g., 106) on opposite sides of the C-arm. These slide rails, together with a mid-length of the C-arm 104, can travail within an outer, shorter C-sleeve (a.k.a. “C-slider housing”) 108. The rails are driven by sprockets (not shown) turned by an electric motor (not shown).

FIGS. 1-7 are identical to the drawings in Grady '205 except: the C-arm support apparatus is referenced by 101 in this application.

One end portion of the C-arm 104 and its slide rails (e.g., 106) are placed behind an image receptor assembly 114 centerline 116. That way, the C-arm can pass by the image receptor assembly 114 without interfering with (i.e., hitting) the assembly 114.

The C-arm projection itself, at either end, or just the end nearest the image receptor, contains balance weights at 116. These weights offset the unbalancing moment of the C-arm mass and source assembly to allow manual motion of the slide axis. These counterweights are similar to the technique found in Grady '941.

Applicant's present drawings include: a rotatable tilt bearing means 120 for pivoting the arc (and C-slider) in a Z-plane around a pivot axis (a.k.a. C-slider axis) 122; and a sliding pivot point means 124 for repositioning the tilt bearing means 120, and pivot axis 122, in the X-Y plane along an arcuate path. The tilt bearing means 120 and the sliding pivot point means 124 combine to create extreme compound angles related to cardiac catheterization (“cath”).

Applicant's illustrated mobile C-arm x-ray device 100 has an x-ray source 102, an image receptor assembly 114, and a C-arm 104 which slides through a fixed C-slider housing 108 mounted onto a portable base 126, the improvement comprising: the C-slider housing 108 is mounted onto the portable base 126 through the rotatable tilt bearing means 120 for pivoting the arc (and C-slider housing 108) in a Z-plane around C-slider rotational axis 122; and, the C-slider rotational axis 122, plus C-slider 108, can be repositioned along an arcuate slot 130 (in the base 126) by the sliding pivot point means 124 from approximately a horizontal position to a position between approximately 60 degrees and 40 degrees below horizontal in a vertical plane.

Sliding pivot point means 124 preferably includes an arcuate pivot carriage guide track 132, aligned with slot 130 and mounted within the base 126. Track 132 and slot 130 have similar arcuate lengths. Those lengths are shown as 60 degrees.

Track 132 has upper and lower surfaces, plus closed ends, that form an internal curved housing for a C-slider pivot carriage 134. Carriage 134 has guide rollers for sliding the carriage 134 within the confines of the guide track housing.

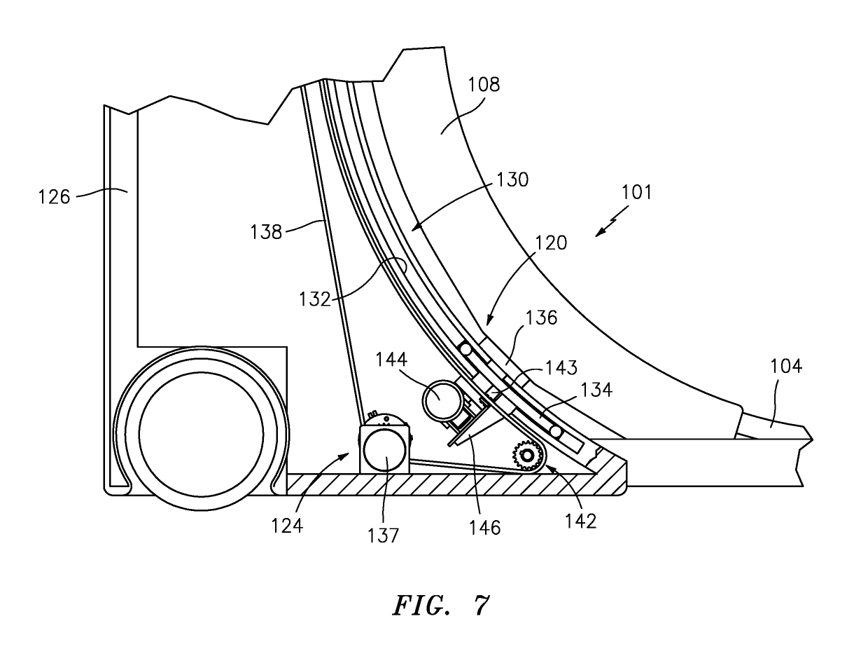

As best shown in FIG. 7, carriage 134 is attached to the C-slider 108 by a rotatable tilt bearing 136 that rides atop a slot (not shown) in guide track 132. A drive gear motor 137 selectively slides the carriage 134 and C-slider 108 by a drive chain 138. The drive chain 138 rides over sprockets 140 and 142, and is fixed to the underside of the carriage by a set screw 143.

As best shown in FIG. 7, tilt bearing means 124 preferably comprises a reversible drive motor 144 removably attached to the rotatable tilt bearing 136 by any suitable means, such as pinning. Motor 144 preferably has a drive shaft which extends through a slot in the bottom of the track 132 housing, up through a hole in the carriage 134. Motor 144 preferably is mounted on a tab 146 that runs in a lower slot of the guide track between the guide rollers. Other mounts and attachments would also do. A user (not shown) can utilize the motor to selectively rotate C-slider housing 108 about the C-slider axis 122.

Without excluding any particular materials or techniques, some examples of Applicant's “non-magnetic” approach are: (i) carbon fiber for C-arm 104, C-arm guide track 132, pivot carriage 134, C-slider housing 108 and portable base 126; (ii) aluminum or brass or plastic for brackets (not shown) and small parts such as drive chain 138, sprockets 140, 142 and set screw 143; (iii) polyethylene for wheel hubs, such as the drive hubs of motors 137, 144, and for bearings, such as the rotatable tilt bearing 136; (iv) Kevlarg belts for rope (not shown) and for the drive gears of motors 137, 144; (v) ceramics for bearings, such as the rotatable tilt bearing 136, and for the C-arm 104, C-arm guide track 132, pivot carriage 134, C-slider housing 108 and portable base 126; or (vi) stainless steel (non-magnetic) for panels, such as the panels of the base 126, for chassis, such as guide track 132 for the rotor (not shown) of the x-ray tube and for fasteners.

This concept uses the absolute minimum of materials which can be magnetized. Other parts, such as the rotor of the x-ray tube and its motor core must be magnetic. Even those perhaps can be eliminated by a stationary anode tube with tungsten embedded in a massive cooled copper casting, as is known from therapy x-ray tubes. The image reception end, of the flat panel design, is in general immune to magnetic effects.

It is the intent of this filing to claim variations on the basic idea, wherein as much steel or iron as possible has been deleted, with the expressed intent to enable use of the x-ray imaging in proximity to an MRI machine by use of materials, which cannot be magnetized, targeted toward fabricating an x-ray system to use with an MRI machine. Both machines are capable of operating simultaneously in the same examination room without interference from the x-ray machine, due to its materials which cannot be magnetized.

FIG. 8 shows the illustrated mobile C-arm x-ray fluoroscopy machine 100, constructed substantially entirely with materials, which cannot be magnetized, in the same examination room 146 as an MRI machine 148. The MRI and C-arm machines can share the same table or stretcher, as shown in FIG. 8. The illustrated table is on a guide track on the floor. By pulling the C-arm device on its wheels away from (perpendicularly) to the table, the table can move through pulleys or gearing (not shown) simultaneously along its guide track next to the MRI. If the table is long enough (e.g., at least 10 feet), the table can transit back and forth, in-line, to bring imaged patient areas into view for both the MRI and C-arm devices. The MRI and fluoroscopy machines are capable of operating simultaneously, due to the materials of the fluoroscopy device which cannot be magnetized.

Though not shown in FIG. 8, there is a travel guide or travel limitation (e.g, a stop) in or on the floor, to prevent inadvertently moving the C-arm too close to the MRI magnet. There will be some residual metal in the C-arm, despite best efforts, and the MRI's magnet could suck the C-arm toward it violently without that travel limitation.

Applicant's invention can be thought of in method terms as follows: building a mobile x-ray machine with materials which cannot be magnetized; and placing the x-ray machine in a same examination room as an MRI machine; whereby the x-ray machine does not interfere with a magnetic field created by the MRI machine and a magnet of the MRI machine does not affect the x-ray machine. Due to the materials of the x-ray machine which cannot be magnetized, both the MRI and x-ray machines are capable of operating simultaneously in the same examination room.

It should be understood by those skilled in the art that obvious structural modifications can be made without departing from the spirit of the invention. For example, while a mobile C-arm support has been referred to, various x-ray positioners or holders such as a parallelogram (see, e.g., U.S. Pat. No. 3,892,967 to Grady et al.) or variation of existing C-arms could be constructed substantially without any materials which can be magnetized. Accordingly, reference should be made primarily to the appended claims rather than the foregoing description to determine the scope of the invention.

I claim:

1. A method comprising:
a. building a mobile x-ray fluoroscopy machine comprised substantially entirely of materials which cannot be magnetized, wherein:
i. the fluoroscopy machine includes an x-ray tube with a rotor and a motor with a core; and
ii. only the rotor and the core can be magnetized;
b. placing the x-ray fluoroscopy machine in a same examination room as a Magnetic Resonance Imaging machine; and
c. whereby, due to the materials which cannot be magnetized, the Magnetic Resonance Imaging machine and the fluoroscopy machine are capable of imaging simultaneously in the room.

2. The method of claim 1 wherein the mobile x-ray fluoroscopy machine includes a mobile C-arm support device.

3. A method comprising:
a. building a mobile x-ray machine comprised substantially entirely of non-magnetic materials which cannot be magnetized;
b. placing the x-ray machine in a same examination room as a Magnetic Resonance Imaging Machine;
c. whereby the x-ray machine, due to the materials which cannot be magnetized, does not interfere with a magnetic field created by a Magnetic Resonance Imaging machine and a magnet of the Magnetic Resonance Imaging machine does not affect the x-ray machine; and
d. whereby, due to the materials of the x-ray machine which cannot be magnetized, the Magnetic Resonance Imaging machine and the fluoroscopy machine are capable of imaging simultaneously in the room.

4. The method of claim 3 wherein the mobile x-ray machine is a mobile x-ray fluoroscopy machine.

5. The method of claim 4 wherein the mobile x-ray fluoroscopy machine includes a mobile C-arm support device.

6. A method comprising:
a. placing an x-ray fluoroscopy machine, comprised substantially entirely of materials which cannot be magnetized, in a same examination room as a Magnetic Resonance Imaging machine;
b. wherein the fluoroscopy machine includes an x-ray tube with a rotor and a motor with a core;
c. wherein only the rotor and the core of the fluoroscopy machine can be magnetized; and
d. whereby, due to the materials which cannot be magnetized, the Magnetic Resonance Imaging machine and the fluoroscopy machine are capable of imaging simultaneously in the room.

7. A method comprising:
a. using an x-ray fluoroscopy machine, comprised substantially entirely of materials which cannot be magnetized, in a same examination room as a Magnetic Resonance Imaging Machine; and
b. whereby the fluoroscopy machine, due to the materials which cannot be magnetized, does not interfere with a magnetic field created by a Magnetic Resonance Imaging machine and a magnet of the Magnetic Resonance Imaging machine does not affect the x-ray fluoroscopy machine; and
c. whereby, due to the materials which cannot be magnetized, the Magnetic Resonance Imaging machine and the fluoroscopy machine are capable of operating simultaneously in the room.
